(19) United States
(12) Patent Application Publication
(10) Pub. No.: US 2004/0185017 A1
(43) Pub. Date: Sep. 23, 2004

(54) Nail varnish composition comprising at least one film-forming gradient copolymer and cosmetic process for making up and/or caring for the nails

(76) Inventors: Nathalie Mougin, Paris (FR); Colette Cazeneuve, Paris (FR); Marco Vicic, Bry S/Marne (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/735,049

(22) Filed: Dec. 15, 2003

Related U.S. Application Data
(60) Provisional application No. 60/458,003, filed on Mar. 28, 2003.

(30) Foreign Application Priority Data
Dec. 13, 2002 (FR) 02 15856

(51) Int. Cl. A61K 7/04

(52) U.S. Cl. 424/61

(57) ABSTRACT

A nail varnish composition comprising, in a cosmetically acceptable medium, at least one film-forming gradient copolymer comprising at least two different monomeric units and exhibiting a mass polydispersity index (PI) of less than or equal to 2.5, the composition being capable of forming a film having an adhesion corresponding to a detachment percentage of less than 45%; a cosmetic process for making up and/or caring for the nails comprising the application to the nails of the cosmetic composition.

This application claims benefit of U.S. Provisional Application No. 60/458,003, filed Mar. 28, 2003.

Disclosed herein are novel cosmetic compositions, for example, make-up compositions, such as nail varnish compositions, comprising specific copolymers, for example, film-forming gradient copolymers, which may be soluble or dispersible in water and/or in organic solvents, and wherein these compositions can exhibit good adhesion properties.

It is known that nail varnish compositions may comprise a film-forming polymer in an organic solvent medium or an aqueous medium. The varnish forms, after drying, a colored or colorless film on the nails and can thus make it possible to embellish or protect the nails against external attack, such as rubbing or scratches. Nail varnishes, however, may frequently exhibit poor hold over time. For example, the film may deteriorate after one or two days, such as by flaking or detaching. Such a deterioration may often occur at the end of the nail. When the varnish is damaged, the user then has to remove the damaged varnish and then apply a fresh application of varnish. The user can also retouch the damaged varnish by partially applying varnish but this type of retouching may not result in an entirely attractive make-up. If the user does nothing, the damaged varnish may harm the attractive appearance of the make-up and may not provide good protection to the nail.

Other nail varnishes, such as easily peelable varnishes or varnishes which can be removed with water, may not confer a very good hold over time either.

Furthermore, the use of a mixture of several polymers, each polymer contributing one of the desired characteristics, with very different chemical natures within the same composition, can cause problems of phase separation within the composition because the chemical natures of the polymers may not necessarily be compatible.

The use of random polymers, for example, conventional acrylic polymers obtained by conventional radical polymerization by random mixing of monomers, may not allow these problems to be solved satisfactorily. This is because the random polymers known in the prior art may exhibit a dispersity in composition of the polymer chains which also can result in a phase separation of the polymers within the formula.

A need thus exists to have available nail varnish compositions which can make it possible to obtain a film deposited on the nails which may exhibit a satisfactory adhesion and hold over time, without exhibiting unsightly flaws, the compositions being, for example, stable and homogeneous.

Thus, disclosed herein is a nail varnish composition that may exhibit at least one of the following properties: good stability and good properties of adhesion and hold over time, for example, good resistance to rubbing, water and/or to flaking.

Further disclosed herein, therefore, is a nail varnish composition comprising, in a cosmetically acceptable medium, at least one film-forming gradient copolymer comprising at least two different monomeric units and exhibiting a mass polydispersity index (PI) of less than or equal to 2.5, wherein the composition is capable of forming a film having an adhesion, measured according to Standard ASTM D 3359-7, corresponding to a detachment percentage of less than 45%.

As used herein, the term “film-forming polymer” is understood to mean a polymer capable of forming, by itself alone or in the presence of an additional agent which is also able to form a film, a continuous film which adheres to the nail at a temperature ranging from 20° C. to 30° C.

The copolymers disclosed herein are gradient copolymers, which comprise at least two different monomeric units and which exhibit a low polydispersity in mass and, for example, a low polydispersity in composition.

Because the copolymers disclosed herein exhibit a low dispersity in composition, and all the chains of the copolymers have virtually the same structures, they are therefore compatible with one another. As a result, the cosmetic compositions comprising these copolymers may not exhibit at least one of the disadvantages and limitations of the prior art compositions.

For example, the at least one film-forming gradient copolymer may exhibit the advantage of being easy to handle in water or in an organic solvent medium while retaining at least one advantageous rheological property.

The polydispersity in mass can be illustrated using the mass polydispersity index (PI) of the at least one film-forming gradient copolymer, which is equal to the ratio of the weight-average molecular mass (Mw) to the number-average molecular mass (Mn).

A low polydispersity in mass reflects copolymers with approximately identical chain lengths, which is the case for the at least one film-forming gradient copolymer disclosed herein.

The at least one film-forming gradient copolymer disclosed herein may, for example, have a mass polydispersity index of less than or equal to 2.5, further, for example, ranging from 1.1 to 2.3, even further, for example, from 1.15 to 2.0, and further, for example, from 1.2 to 1.9 or 1.8.

Furthermore, the weight-average molecular mass of the at least one film-forming gradient copolymer may range, for example, from 5 000 g/mol to 1 000 000 g/mol, further, for example, from 5 500 g/mol to 800 000 g/mol and, even further, for example, from 6 000 g/mol to 500 000 g/mol.

Further, for example, the number-average molecular mass of the at least one film forming gradient copolymer can range from 5 000 g/mol to 1 000 000 g/mol, further, for example, from 5 500 g/mol to 800 000 g/mol and, even further, for example, from 6 000 g/mol to 500 000 g/mol.

The weight-average molecular masses (Mw) and the number-average molecular masses (Mn) are determined by gel permeation liquid chromatography (GPC), (eluent THF, calibration curve established with linear polystyrene standards, refractometer detector).

The at least one film-forming gradient copolymer disclosed herein may also, for example, exhibit a low dispersity in composition. This means that all the chains of the copolymers are composed of a sequence of monomers which are approximately the same and are therefore homogeneous in composition.

Liquid adsorption chromatography (“LAC”) may be used to show that all the chains of the copolymers have a similar composition. LAC makes it possible to separate the chains of copolymers not according to their molecular weight but according to their polarity. The polarity reflects the chemical composition of the polymers constituting the material, provided that the monomers are known.

Reference may be made to the publication Macromolecules (2001), 34, 2667, which describes the LAC technique.

The polydispersity in composition can be defined, for example, from the adsorption chromatography (LAC) curve (curve representing the proportion of polymers as a function of the elution volume): if “V^{1/2} min” is used to denote the minimum value of the elution volume at mid-height of the curve and if “V^{1/2} max” is used to denote the maximum value of the elution volume at mid-height of the curve, the polydispersity in composition is regarded as low if the difference (V^{1/2} max−V^{1/2} min) is less than or equal to 3.5, for example, ranging from 1 to 2.8, and further, for example, ranging from 1.2 to 2.5.

Furthermore, the LAC curve can be defined by a gaussian curve of formula:

$$y = \frac{A}{w \sqrt{\frac{π}{2}}} \times {ⅇ}^{- 2 \frac{{( x - {x}_{0} )}^{2}}{{w}^{2}}} + {y}_{o}$$

wherein:

x₀ is the value of x (elution volume) at the center of the peak;

w is equal to twice the standard deviation of the gaussian distribution (i.e. 2σ) or alternatively corresponds to approximately 0.849 times the width of the peak at mid-height;

A represents the area under the peak; and

yₒ is the value of y corresponding to x₀.

The polydispersity in composition can also be defined by the value w as defined above.

For example, the value w can range, for example, from 1 to 3, further, for example, from 1.1 to 2.3 and, even further, for example, from 1.1 to 2.0.

The at least one film-forming gradient copolymer disclosed herein can be obtained by living or pseudo-living polymerization.

For the record, it should be remembered that living polymerization is a polymerization in which the growth of the polymer chains only stops when the monomer disappears. The number-average molecular mass (Mn) increases with the conversion. Anionic polymerisation is a typical example of living polymerization. Such polymerizations result in copolymers having a low dispersity in mass, that is to say in polymers with a mass polydispersity index (PI), for example, of less than 2.

Pseudo-living polymerization is associated with controlled radical polymerization. For example, the main types of controlled radical polymerization may be:

radical polymerization controlled by nitroxides. For example, Patent Application Nos. WO 96/24620 and WO 00/71501 disclose the tools of this polymerization and their use, as do the papers published by Fischer (Chemical Reviews, 2001, 101, 3581), by Tordo and Gnanou (J. Am. Chem. Soc., 2000, 122, 5929) and by Hawker (J. Am. Chem. Soc., 1999, 121, 3904);

atom transfer radical polymerization, disclosed, for example, in Patent Application No. WO 96/30421 and which proceeds by the reversible insertion of an organometallic complex in a carbon-halogen bond; and

radical polymerization controlled by sulphur derivatives of xanthate, dithioester, trithiocarbonate or carbamate, disclosed, for example, in Patent Application Nos. FR 2 821 620, WO 98/01478, WO 99/35177, WO 98/58974, WO 99/31144 and WO 97/01478 and in the publication by Rizzardo et al. (Macromolecules, 1998, 31, 5559).

Controlled radical polymerization are polymerizations in which the secondary reactions which usually result in the disappearance of propagating entities (termination or transfer reaction) are rendered highly improbable in comparison with the propagation reaction by virtue of an agent for controlling the free radicals. The disadvantage of this method of polymerization lies in the fact that, when the concentrations of free radicals become high in comparison with the concentration in monomer, the secondary reactions again become determining and tend to broaden the distribution of the masses.

By virtue of these polymerization methods, the polymer chains of the at least one film-forming gradient copolymer disclosed herein grow simultaneously and therefore incorporate at each instant the same ratio of comonomers. All the chains therefore have the same structures or similar structures, resulting in a low dispersity in composition. These chains may also have a low mass polydispersity index.

As used herein, “gradient copolymers” are copolymers exhibiting a change in the ratio of the various monomer units all along the chain. The distribution in the polymeric chains of the comonomeric units depends on the change during the synthesis of the relative concentrations of the comonomeric units.

The at least one film-forming gradient copolymer comprises at least two different monomeric units, the concentration of which along the polymer chain can change gradually and in a systematic and predictable way.

This means that all the polymer chains comprise at least one monomeric unit Mi, such that there is a nonzero probability of encountering this monomer unit Mi along the chain, regardless of the normalized position x on the polymer chain.

One of the characteristics which makes it possible to define the at least one film forming gradient copolymer is the fact that, at any instant in the polymerization, all the chains are subjected to the presence of the combination of all the monomers. Thus, in the reaction medium, the concentration of each monomer is always nonzero at any instant in the polymerisation.

This makes it possible to distinguish the least one film-forming gradient copolymer from conventional block polymers in which the change in the monomeric units along the polymer chain is not systematic; for example, for an AB diblock, within the A block, the concentration of the other monomeric unit B is always zero.

In the case of random polymers, the change in the monomeric units along the polymer chain will not be gradual, systematic and predictable either. As illustrated by the diagram below, a random polymer obtained by conventional radical polymerization of two monomers is distinguished from a gradient copolymer by the distribution of the monomeric units, which is not identical over all the chains, and by the length of the chains, which is not identical for all the chains.

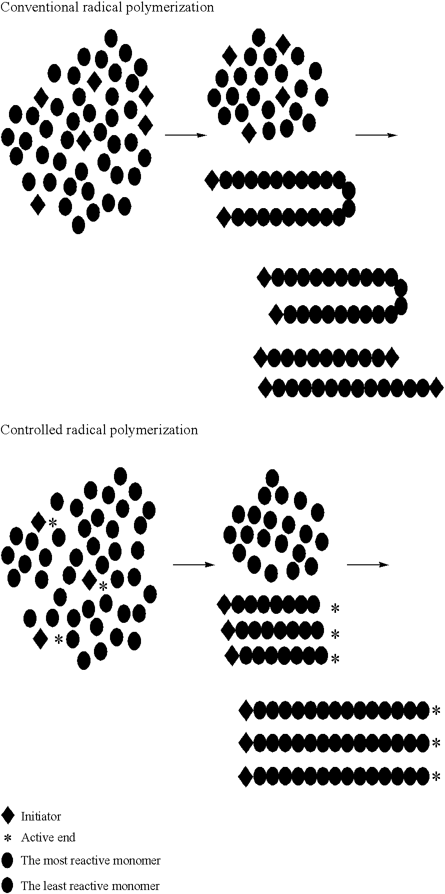

For a theoretical description of gradient copolymers, reference may be made to the following publications:

T. Pakula et al., Macromol. Theory Simul., 5, 987-1006 (1996);

A. Aksimetiev et al., J. of Chem. Physics, 111, No. 5;

M. Janco, J. Polym. Sci., Part A: Polym. Chem. (2000), 38(15), 2767-2778;

M. Zaremski et al., Macromolecules (2000), 33(12), 4365-4372;

K. Matyjaszewski et al., J. Phys. Org. Chem. (2000), 13(12), 775-786;

Gray, Polym. Prepr. (Am. Chem. Soc., Div. Polym. Chem.) (2001), 42(2), 337-338;

K. Matyjaszewski, Chem. Rev. (Washington, D.C.) (2001), 101(9), 2921-2990.

Among gradient copolymers, it is possible to distinguish natural gradient copolymers and artificial gradient copolymers.

A natural gradient copolymer is a gradient copolymer synthesized as a batch from a starting mixture of comonomers. The distribution in the chain of the various monomers depends upon the relative reactivity of the monomers and upon the starting concentrations of the monomers. These copolymers constitute the simplest class of gradient copolymers as it is the starting mixture which defines the final product property.

An artificial gradient copolymer is a copolymer for which the concentration of monomers during the synthesis is varied by a processing expedient. In this case, a mixture of monomers is changed to another in the chain due to a sudden and abrupt change in the monomers in the reaction medium (by removing the first mixture or adding at least one new monomer). It is even possible for at least one of the monomers therein to completely disappear, to the advantage of one or more others.

The gradient is determined experimentally by measuring, during polymerization, the chemical composition of the polymer. This measurement is performed indirectly by determining the change in the content of the various monomers at any instant. It can be performed by NMR and UV spectroscopy, for example.

This is because, for the polymers prepared by living or pseudo-living polymerization, the length of the chains is linearly related to the conversion.

By withdrawing a sample of the polymerization solution at various instants in the polymerization and by measuring the difference in content of each monomer, the composition of the gradient is thus determined.

The distribution of the compositions of the chains is narrow in the gradient polymer. For example, no overlap exists between the peak of the gradient copolymer and those of the respective homopolymers. This means that the material obtained under gradient conditions is composed of polymer chains with the same composition whereas, in conventional random polymerization, different kinds of chain coexist, including those of the respective homopolymers.

It is possible to characterize gradient copolymers by a vector characteristic of each copolymer.

This is because, knowing that there exists an infinity of polymers characterized by a given chemical composition, to specify a polymer it is possible to describe the distribution of monomeric units along the chain. This involves a description comprising several variables. This vector is a point in space of the chemical compositions.

The exact term is that G is a vector, the coordinates of which are the concentrations of the monomeric units along the polymer chain. These concentrations are defined by the rules of the reactivity coefficients of each of the monomers and therefore are related to the concentration of the free monomers during the synthesis: from the moment that the monomer is not in zero concentration in the reaction mixture, it is not in zero concentration in the polymer.

It is therefore possible to characterize gradient copolymers by the function G(x) which defines the composition gradient:

\vec{G}(x)=Σ\vec{[Mi](x)}

wherein:

x is a normalized position on the polymer chain and

[Mi](x) is the relative concentration, in this position x, of the monomeric unit Mi, expressed in mol %.

The function G(x) therefore locally describes the composition of the gradient copolymer.

Two copolymers can have an equivalent composition overall but very different local distributions of the monomeric units and therefore different gradients.

For example, in the case of a (50/50) AB diblock, the function [A] has a value of 1 up to x=½, and then 0 subsequently.

The factors which determine the gradient are, first, the relative reactivity coefficients of each monomeric unit (referred to as rᵢ for the monomeric unit Mi), which depend mainly on the type of synthesis process employed (homogeneous, dispersed) and on the solvents and, secondly, the starting concentrations of each of the monomers and the possible additions of monomers during polymerization.

Thus, if, by way of example, a gradient copolymer of styrene (M1) having a relative reactivity coefficient r₁=0.418 and of methacrylic acid (M2), with r₂=0.6, in a homogeneous polymerisation system is considered, the variation in the starting concentrations of styrene and of methacrylic acid makes it possible to obtain different gradient copolymers having chains with completely different structures.

When the starting concentration of methacrylic acid is 10% by weight, a very weak gradient copolymer is obtained in the end for which nanostructure formation cannot be expected. When the starting concentration is 20% by weight, a gradient copolymer is obtained which has a hydrophilic “head” and a hydrophobic “tail” with a sufficiently pronounced gradient to result in nanostructure formation. When this concentration is 50% by weight, as the monomers are isoreactive under these conditions, the copolymer obtained is an alternating copolymer.

Although the copolymers described are all gradient copolymers of styrene and of methacrylic acid, the difference in starting concentration of the monomers results in chains with completely different structures which confer different properties on the copolymers. This example therefore illustrates the importance of the starting monomer compositions on the arrangement along the chain of the various monomers.

In the case of a styrene/methacrylic acid gradient copolymer, the various polymers obtained can be represented diagrammatically thus, the white balls corresponding to styrene while the dark balls correspond to methacrylic acid:

10% of Methacrylic Acid Initially:

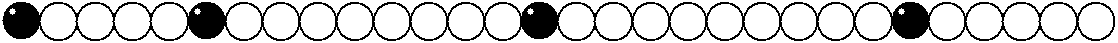

Very weak gradient copolymer for which nanostructure formation cannot be expected.

20% of Methacrylic Acid Initially:

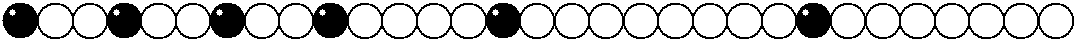

Copolymer with a hydrophilic “head” and hydrophobic “tail” with a sufficiently pronounced gradient to result in nanostructure formation.

50% of Methacrylic Acid Initially:

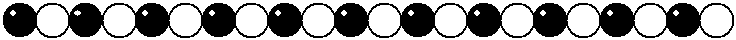

As the monomers are isoreactive under these conditions, the copolymer obtained is an alternating copolymer.

The structure of these polymers can be determined by the disappearance of the methacrylic acid as a function of the degree of conversion.

The at least one film-forming gradient copolymer disclosed herein comprises at least two different monomeric units which can each be present in an amount ranging, for example, from 1 to 99% by weight, relative to the weight of the final copolymer, further, for example, in an amount ranging from 2 to 98% by weight, relative to the weight of the final copolymer, and even further, for example, ranging from 5 to 95% by weight, relative to the weight of the final copolymer.

The at least one film-forming gradient copolymer may, for example, comprise at least one hydrophilic monomeric unit.

The at least one hydrophilic monomeric unit can be present in an amount ranging, for example, from 1 to 99% by weight, relative to the total weight of the copolymer, further, for example, in an amount ranging from 2 to 70% by weight, relative to the total weight of the copolymer, further, for example, ranging from 3 to 50% by weight, relative to the total weight of the copolymer, and further, for example, ranging from 4 to 30% by weight, relative to the total weight of the copolymer, and even further, for example, ranging from 5 to 25% by weight, relative to the total weight of the copolymer.

As used herein, the term “hydrophilic monomeric unit” means units or residues of at least one monomer, the homopolymers of which are soluble or dispersible in water or whose ionic form is soluble or dispersible in water.

A homopolymer is “water-soluble” if it forms a clear solution when it is in solution in water at 1% by weight at 25° C.

A homopolymer is “water-dispersible” if, in water at 1% by weight at 25° C., it forms a stable suspension of fine particles, such as spherical particles. The mean size of the particles constituting the dispersion is less than 1 μm and may, for example, range from 5 to 400 nm, and further, for example, range from 10 to 250 nm. These particle sizes are measured by a light scattering technique.

The homopolymer(s) formed from the at least one hydrophilic monomeric unit disclosed herein can have awith a Tg of greater than or equal to 20° C., for example, of greater than or equal to 50° C., but can optionally have a Tg of less than or equal to 20° C.

The at least one film-forming gradient copolymer can also comprise at least one hydrophobic monomeric unit, for example, at least one hydrophobic monomeric unit capable of being rendered hydrophilic after polymerization. The at least one hydrophobic monomeric unit can be rendered hydrophilic, for example, by chemical reaction, such as by hydrolysis, or by chemical modification, for example, of an ester functional group, or by incorporation of chains comprising at least one hydrophilic unit, for example, a carboxylic acid.

The at least one hydrophobic monomeric unit can be present in an amount ranging, for example, from 1 to 99% by weight, relative to the total weight of the copolymer, further, for example, from 30 to 98% by weight, relative to the total weight of the copolymer, further, for example, from 50 to 97% by weight, relative to the total weight of the copolymer, further, for example, from 70 to 96% by weight, relative to the total weight of the compolymer, and even further, for example, from 75 to 95% by weight, relative to the total weight of the copolymer.

The homopolymer(s) formed from the at least one hydrophobic monomeric unit can, for example, have a Tg of greater than or equal to 20° C., for example, greater than or equal to 30° C., but can optionally have a Tg of less than or equal to 20° C.

The at least one film-forming gradient copolymer disclosed herein may, for example, comprise at least one monomeric unit, the homopolymer of which has a Tg of less than or equal to 20° C., for example, ranging from −150° C. to 20° C., further, for example, ranging from −130° C. to 18° C. and, even further, for example, ranging from −120° C. to 15° C.

The at least one monomeric unit with a Tg≦20° C. as disclosed above can be present in the at least one film-forming gradient copolymer in an amount ranging, for example, from 1 to 99% by weight, relative to the total weight of the copolymer, further, for example, ranging from 20 to 90% by weight, relative to the total weight of the copolymer, further, for example, ranging from 30 to 85% by weight, relative to the total weight of the copolymer, and further, for example, ranging from 50 to 75% by weight, relative to the total weight of the copolymer.

The at least one monomeric unit with a Tg≧20° C. can therefore be present in an amount ranging from 1 to 99% by weight, relative to the total weight of the copolymer, further, for example, from 10 to 80% by weight, relative to the total weight of the copolymer, further, for example, ranging from 15 to 70% by weight, relative to the total weight of the copolymer, and even further, for example, ranging from 25 to 50% by weight, relative to the total weight of the copolymer.

As used herein, the term “monomer with a Tg” means monomers, the homopolymers of which have such a Tg, measured according to the method described below.

As used herein, the Tg (or glass transition temperature) is measured according to Standard ASTM D3418-97 by differential scanning calorimetry (DSC) on a calorimeter over a temperature ranging from −100° C. to +150° C. at a heating rate of 10° C./min in aluminium crucibles with a capacity of 150 μl.

In one embodiment, the at least one film-forming gradient copolymer disclosed herein comprises three different monomeric units which can each be present in an amount ranging, for example, from 5 to 90% by weight, relative to the total weight of the copolymer, for example, from 7 to 86% by weight, relative to the total weight of the copolymer.

For example, the at least one film-forming gradient copolymer can comprise 5 to 25% by weight of a first monomer, relative to the total weight of the copolymer; 5 to 25% by weight of a second monomer, relative to the total weight of the copolymer; and 50 to 90% by weight of a third monomer, relative to the total weight of the copolymer.

For example, the at least one film-forming gradient copolymer disclosed herein can comprise 5 to 25% by weight of a hydrophilic monomer unit, relative to the total weight of the copolymer; 50 to 90% by weight of a monomeric unit, the homopolymer of which has a Tg of less than or equal to 20° C.; relative to the total weight of the copolymer; and 5 to 25% by weight of an additional monomer unit, relative to the total weight of the copolymer.

For example, hydrophilic monomers (i.e., hydrophilic monomeric units) useful in the composition disclosed herein may be chosen from the following:

amino(C₁-C₄ alkyl) (meth)acrylate derivatives, for example, N,N-di(C₁-C₄ alkyl)amino(C₁-C₆ alkyl) (meth)acrylates, such as N,N-dimethylaminoethyl methacrylate (MADAME) or N,N-diethylaminoethyl methacrylate (DEAMEA);

N,N-di(C₁-C₄ alkyl)(meth)acrylamides and N,N-di(C₁-C₄ alkyl)amino(C₁-C₆ alkyl)(meth)acrylamides, such as N,N-dimethylacrylamide, N,N-dimethylamino-propylacrylamide (DMAPA) or N,N-dimethylaminopropylmethacrylamide (DMAPMA),

di(C₁-C₈ alkyl)allylamines, such as dimethyldiallylamine;

vinylamine; and

vinylpyridines, for example, 2-vinylpyridine or 4-vinylpyridine; and the salts thereof with inorganic acids or organic acids and the quatemized forms thereof.

For example, the inorganic acids may be chosen from sulphuric acid, hydrochloric acid, hydrobromic acid, hydriodic acid, phosphoric acid, and boric acid.

Further, for example, the organic acids may be chosen from acids comprising at least one group chosen from carboxyl, sulpho and phosphono groups. The organic acids may, for example, be chosen from linear, branched and cyclicaliphatic acids and from aromatic acids. These acids can additionally comprise at least one heteroatom chosen from O and N, for example, the at least one heteroatom may be in the form of a hydroxyl group.

An example of an acid with an alkyl group is acetic acid or propionic acid. An example of a polyacid is terephthalic acid. Examples of hydroxyacids are citric acid and tartaric acid.

The quaternizing agents can be chosen from alkyl halides, such as methyl bromide, or alkyl sulphates, such as methyl sulphate, or propane sultone.

Other useful hydrophilic monomers may, for example, be chosen from:

ethylenic carboxylic acids, for example, mono- or dicarboxylic acids, such as acrylic acid, methacrylic acid, crotonic acid, itaconic acid, fumaric acid or maleic acid;

carboxylic anhydrides comprising at least one vinyl bond, such as maleic anhydride;

ethylenic sulphonic acids, such as styrenesulphonic acid, acrylamidopropanesulphonic acid, and the salts thereof;

vinylbenzoic acid, vinylphosphonic acid and the salts thereof; and

the potassium salt of 3-(acryloyloxy)propanesulphonic acid and the compound of formula CH₂═CHCOOCH₂OCH₂(OH)CH₂SO₃⁻Na⁺.

The neutralizing agent can be chosen from inorganic bases, such as LiOH, NaOH, KOH, Ca(OH)₂ or NH₄OH; and organic bases, for example a primary, secondary or tertiary amine, for example, optionally hydroxylated alkylamines, such as dibutylamine, triethylamine or stearamine, or 2-amino-2-methylpropanol, monoethanolamine, diethanolamine or stearamidopropyldimethylamine.

Even further, for example, the at least one hydrophilic monomeric unit may also be chosen from:

amides of unsaturated carboxylic acids, such as acrylamide or methacrylamide and the N-substituted derivatives thereof, for example N-(C₁-C₄ alkyl)(meth)acrylamides, such as N-methylacrylamide, or N,N-di(C₁-C₄ alkyl)(meth)acrylamides, such as N,N-dimethyl-acrylamide;

hydroxyalkyl (meth)acrylates, for example, those in which the alkyl group comprises from 2 to 4 carbon atoms, such as hydroxyethyl (meth)acrylate;

(meth)acrylates of polyethylene glycol (5 to 100 EO) or of glycol which are optionally substituted on their end functional group by at least one group chosen from alkyl, phosphate, phosphonate and sulphonate groups, for example glyceryl acrylate, methoxypolyethylene glycol (8 or 12 EO) (meth)acrylate or hydroxypolyethylene glycol (meth)acrylate;

alkoxyalkyl (meth)acrylates, such as ethoxyethyl (meth)acrylate;

polysaccharide (meth)acrylates, such as sucrose acrylate;

vinylamides, such as vinylacetamide; optionally cyclic vinylamides, for example, vinyllactams, such as N-vinylpyrrolidone or N-vinylcaprolactam; and

vinyl ethers, such as vinyl methyl ether.

Further, for example, the at least one hydrophilic monomer unit may be chosen from:

methacrylamidopropoxytrimethylammonium betaine;

N, N-dimethyl-N-methacryloyloxyethyl-N-(3-sulphopropyl)ammonium betaine;

3-methacryloylethoxycarbonylpyridinium;

the compound of formula:

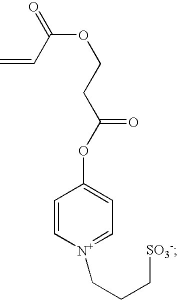

and

4-vinylpyridiniumsulphopropyl betaine of formula:

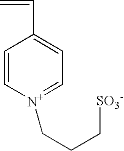

The at least one hydrophilic monomeric unit may, for example, be chosen from N,N-dimethylaminoethyl methacrylate (MADAME), acrylic acid, methacrylic acid, crotonic acid, styrenesulphonic acid, acrylamidopropanesulphonic acid, dimethylaminopropylmethacrylamide (DMAPMA), styrenesulphonate, hydroxyethyl acrylate, glyceryl acrylate, ethoxyethyl methacrylate, ethoxyethyl acrylate, methoxypolyethylene glycol (8 or 12 EO) (meth)acrylate, hydroxypolyethylene glycol (meth)acrylate, N-vinylpyrrolidone, N-vinylcaprolactam, acrylamide and N,N-dimethylacrylamide.

With respect to0 the at least one hydrophobic monomeric unit capable of being rendered hydrophilic, for example, by hydrolysis, mention may be made of C₁-C₄ alkyl (meth)acrylates, such as tert-butyl (meth)acrylate or ethyl (meth)acrylate, which result in the production of (meth)acrylic acid after hydrolysis.

With respect to the at least one monomeric unit, the homopolymer of which has a Tg of less than or equal to 20° C., some of which may be hydrophilic, those which can be used in the composition disclosed herein may, for example, be chosen from:

ethylenic hydrocarbons comprising from 2 to 10 carbons, such as ethylene, isoprene or butadiene;

acrylates of formula CH₂═CHCOOR₁, wherein R₁ is chosen from saturated and unsaturated, linear and branched, hydrocarbonaceous groups comprising from 1 to 12 carbon atoms with the exception of the tert-butyl group, wherein at least one heteroatom chosen from O, N, S and Si is optionally inserted, it being possible in addition for the hydrocarbonaceous group, of R₁, to be optionally substituted by at least one substituent chosen from hydroxyl groups and from halogen atoms chosen from Cl, Br, I and F atoms;

For example, the R₁ groups may be chosen from methyl, ethyl, propyl, butyl, isobutyl, hexyl, ethylhexyl, octyl, lauryl, isooctyl, isodecyl, hydroxyethyl, hydroxypropyl, methoxyethyl, ethoxyethyl, methoxypropyl, ethylperfluorooctyl and propylpolydimethylsiloxane groups;

R₁ can also be chosen from groups of formula —(R″)ₓ—(OC₂H₄)ₙ—OR′″, wherein x=0 or 1, R″ is chosen from saturated and unsaturated, linear and branched, hydrocarbonaceous groups comprising from 1 to 12 carbons, n is a number ranging from 5 to 100 and R′″ is chosen from H and CH₃, and, for example, a methoxy(PEO)8-stearyl group;

methacrylates of formula: CH₂═C(CH₃)—COOR₂ wherein R₂ is chosen from saturated and unsaturated, linear and branched, hydrocarbonaceous groups comprising from 3 to 12 carbons wherein at least one heteroatom chosen from O, N, S and Si is optionally inserted, it being possible in addition for the hydrocarbonaceous groups to be optionally substituted with at least one substituent chosen from hydroxyl groups and from halogen atoms chosen from Cl, Br, I, and F atoms; for example, the R₂ groups may be chosen from hexyl, ethylhexyl, octyl, lauryl, isooctyl, isodecyl, dodecyl, methoxyethyl, methoxypropyl, ethoxyethyl, ethylperfluorooctyl and propylpolydimethylsiloxane groups;

R₂ can also be chosen from groups of formula —(R″)ₓ—(OC₂H₄)ₙ—OR′″, wherein x=0 or 1, R″ is chosen from saturated and unsaturated, linear and branched, hydrocarbonaceous groups comprising from 1 to 12 carbon atoms, n is a number ranging from 5 to 100 and R′″ is chosen from H and CH₃, and, for example, a methoxy(PEO)8-stearyl group;

N- or N,N-substituted derivatives of unsaturated C₁₋₁₂ carboxylic acid amides, for example, N-(C₁₋₁₂ alkyl)(meth)acrylamides, such as N-octylacrylamide;

vinyl esters of formula: R₃—CO—O—CH═CH₂, wherein R₃ is chosen from linear and branched alkyl groups comprising from 2 to 12 carbons, for example, vinyl propionate, vinyl butyrate, vinyl ethylhexanoate, vinyl neononanoate and vinyl neododecanoate; and

vinyl alkyl ethers, the alkyl group comprising from 1 to 12 carbons, such as methyl vinyl ether and ethyl vinyl ether.

The at least one monomeric unit the homopolymer of which has a Tg of less than or equal to 20° C. may, for example, be chosen from:

isoprene and butadiene;

methyl acrylate, ethyl acrylate, isobutyl acrylate, n-butyl acrylate, ethylhexyl acrylate, methoxyethyl acrylate, ethoxyethyl acrylate or hydroxypolyethylene glycol acrylate;

ethoxyethyl methacrylate, hexyl methacrylate, ethylhexyl methacrylate and hydroxypolyethylene glycol methacrylate;

N—(C₆₋₁₂ alkyl)(meth)acrylamides, such as N-octylacrylamide; and

vinyl esters of formula: R₃—CO—O—CH═CH₂, wherein R₃ is chosen from linear and branched alkyl groups comprising from 6 to 12 carbons, for example, vinyl neononanoate and vinyl neododecanoate.

For example, the at least one monomeric unit the homopolymer of which has a Tg of greater than or equal to 20° C., some of which may be hydrophilic, may be chosen from:

vinyl compounds of formula: CH₂═CH—R₄, wherein R₄ is chosen from a hydroxyl group; an

NH—C(O)—CH₃ group; an —OC(O)—CH₃ group; a C₃ to C₈ cycloalkyl group; a C₆ to C₂₀ aryl group; a C₇ to C₃₀ aralkyl group (C₁ to C₄ alkyl group); a 4- to 12-membered heterocyclic group comprising at least one heteroatom chosen from O, N and S; and heterocyclylalkyl (C₁ to C₄ alkyl) groups, such as a furfuryl group; it being possible for the cycloalkyl, aryl, aralkyl, heterocyclic or heterocyclylalkyl groups to be optionally substituted with at least one substituent chosen from hydroxyl groups, halogen atoms and linear and branched C₁ to C₄ alkyl groups wherein at least one heteroatom chosen from O, N, S and P is optionally inserted, it being possible in addition for the alkyl groups to be optionally substituted by at least one substituent chosen from hydroxyl groups and Cl, Br, I, F, and Si atoms.

For example, the vinyl monomeric units may be chosen from vinylcyclohexane, styrene and vinyl acetate;

acrylates of formula CH₂═CH—COOR₅, wherein R₅ is chosen from a tert-butyl group; a C₃ to C₈ cycloalkyl group; a C₆ to C₂₀ aryl group; a C₇ to C₃₀ aralkyl group (C₁ to C₄ alkyl group); a 4- to 12-membered heterocyclic group comprising at least one heteroatom chosen from O, N and S; and heterocyclylalkyl (C₁ to C₄ alkyl) groups, such as a furfuryl group; it being possible for the cycloalkyl, aryl, aralkyl, heterocyclic or heterocyclylalkyl groups to be optionally substituted by at least one substituent chosen from hydroxyl groups, halogen atoms and linear and branched C₁ to C₄ alkyl groups wherein at least one heteroatom chosen from O, N, S and P is optionally inserted, it being possible in addition for the alkyl groups to be optionally substituted by at least one substituent chosen from hydroxyl groups, from halogen atoms chosen from Cl, Br, I, and F atoms, and from Si atoms. For example, the acrylate monomeric units may be chosen from t-butylcyclohexyl acrylate, tert-butyl acrylate, t-butylbenzyl acrylate, furfuryl acrylate and isobornyl acrylate;

methacrylates of formula CH₂═C(CH₃)—COOR₆, wherein R₆ is chosen from linear and branched C₁ to C₄ alkyl groups, such as methyl, ethyl, propyl and isobutyl groups, it being possible in addition for the alkyl groups to be optionally substituted by at least one substituent chosen from hydroxyl groups, from halogen atoms chosen from Cl, Br, I, and F atoms, and from Si atoms; a C₃ to C₈ cycloalkyl group; a C₆ to C₂₀ aryl group; a C₇ to C₃₀ aralkyl group (C₁ to C₄ alkyl group); a 4- to 12-membered heterocyclic group comprising at least one heteroatom chosen from O, N and S; and a heterocyclylalkyl (C₁ to C₄ alkyl) group, such as a furfuryl group; it being possible for the cycloalkyl, aryl, aralkyl, heterocyclic or heterocyclylalkyl groups to be optionally substituted by at least one substituent chosen from hydroxyl groups, halogen atoms and linear and branched C₁ to C₄ alkyl groups wherein at least one heteroatom chosen from O, N, S and P is optionally inserted, it being possible in addition for the alkyl groups to be optionally substituted by at least one substituent chosen from hydroxyl groups and from halogen atoms chosen from Cl, Br, I and F atoms.

For example, the methacrylate monomeric units may be chosen from methyl methacrylate, ethyl methacrylate, n-butyl methacrylate, isobutyl methacrylate, t-butylcyclohexyl methacrylate, t-butylbenzyl methacrylate, methoxyethyl methacrylate, methoxypropyl methacrylate and isobornyl methacrylate;

(meth)acrylamides of formula: CH₂═C(R′)—CO—NR₇R₈, wherein R₇ and R₈, which may be identical or different, are each chosen from a hydrogen atom and linear and branched alkyl groups comprising from 1 to 12 carbon atoms, such as a n-butyl, t-butyl, isopropyl, isohexyl, isooctyl or isononyl group, and R′ is chosen from a hydrogen atom and a methyl group.

For example, the (meth)acrylamide monomeric units may be chosen from N-butylacrylamide, N-(t-butyl)acrylamide, N-isopropylacrylamide, N,N-dimethylacrylamide and N,N-dibutylacrylamide.

Further, for example, the at least one monomeric unit, the homopolymer of which has a Tg of greater than or equal to 20° C. may be chosen from:

furfuryl acrylate, isobornyl acrylate, tert-butyl acrylate, tert-butylcyclohexyl acrylate and tert-butylbenzyl acrylate;

methyl methacrylate, n-butyl methacrylate, ethyl methacrylate and isobutyl methacrylate;

styrene and styrenesulphonate; and

vinyl acetate and vinylcyclohexane.

A person skilled in the art will know how to choose the monomers and their amounts according to the results desired, taking as basis his or her general knowledge, for example, their knowledge of the relative reactivity of each monomer.

Thus, if a copolymer having hydrophilic units in the heart of a polymer chain is desired, a difunctional initiator and a mixture of monomers such that the reactivity of the hydrophilic monomers is greater than that of the other monomers will, for example, be chosen.

Furthermore, it has been found that the preparation processes employed make it possible to adjust and modify the Tg value or values of the copolymer and thus to obtain a gradient copolymer having at least one given Tg value.

The at least one film-forming gradient copolymer disclosed herein can be prepared by a person skilled in the art according to the following procedure:

1) A mixture of the various monomers is prepared, optionally in a solvent, for example, in a stirred reactor. A radical polymerization initiator and an agent for controlling the polymerization is added. The mixture is, for example, placed under a gas atmosphere which is inert with respect to radical polymerization, such as nitrogen or argon.

The optional polymerization solvent may, for example, be chosen from alkyl acetates; such as butyl acetate or ethyl acetate; aromatic solvents, such as toluene; ketone solvents, such as methyl ethyl ketone; and alcohols, such as ethanol. In the case where the mixture of monomers is miscible with water, the latter can advantageously be used as a solvent or a cosolvent.

2) The mixture is brought with stirring to the desired polymerization temperature. This temperature may, for example, range from 10° C. to 160° C., further, for example, from 25° C. to 130° C.

The choice of the polymerization temperature may, for example, be optimized in relation to the chemical composition of the monomer mixture. Thus, monomers having very high propagation kinetic constants and a weaker affinity for the control agent will, for example, be polymerized at a low temperature (for example, in the case of a high proportion of methacrylic derivatives, polymerization at a temperature ranging from 25° to 80° C. can be used).

3) The polymerization medium may be optionally modified during the polymerization, before 90% conversion of the starting monomers is achieved, by further addition of at least one monomer, for example, of the starting mixture. This addition can be carried out in various ways, which can range from the sudden addition all at once to the continuous addition over the entire duration of the polymerization.

4) The polymerization is stopped when the desired degree of conversion is achieved. The overall composition of the copolymer depends on this conversion. The polymerization may, for example, be stopped after having achieved at least 50% conversion, even further, for example, after having achieved at least 90% conversion.

5) The possible residual monomers can be removed by any known method, such as by evaporation or by addition of an amount of a conventional polymerization initiator, such as peroxide or azo derivatives.

In one embodiment, the at least one agent for controlling the polymerization capable of being employed is chosen from nitroxides of formula (I):

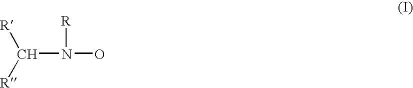

wherein:

R and R′, which may be identical or different, are each chosen from linear and branched, saturated hydrocarbonaceous (alkyl) groups comprising from 1 to 40 carbon atoms which are optionally substituted by at least one group chosen from —OR₃, —COOR₃ and —NHR₃ (wherein R₃ is chosen from H and linear and branched, saturated hydrocarbonaceous (alkyl) groups comprising from 1 to 40 carbon atoms), it being possible in addition for R and R′ to be connected so as to form a ring.

For example, R and R′, which may be identical or different, are each chosen from linear and branched alkyl groups comprising from 1 to 12 carbon atoms, such as methyl, ethyl, propyl, n-butyl, isobutyl, tert-butyl and pentyl groups. Further, for example, R and R′ are both tert-butyl groups;

R″ is chosen from monovalent groups with a molar mass (Mw) of greater than 16 g/mol, for example, R″ is chosen from phosphorus-comprising groups of formula:

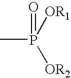

wherein R₁ and R₂, which may be identical or different, are each chosen from linear and branched, saturated hydrocarbonaceous (alkyl) groups comprising from 1 to 40 carbon atoms which are optionally substituted with at least group chosen from —OR₃, —COOR₃ and —NHR₃ (wherein R₃ is chosen from H and linear and branched, saturated hydrocarbonaceous (alkyl) groups comprising from 1 to 40 carbon atoms), it being possible in addition for R₁ and R₂ to be connected so as to form a ring.

For example, R₁ and R₂, which may be identical or different, are each chosen from linear and branched alkyl groups comprising from 1 to 12 carbon atoms, for example, methyl, ethyl, propyl, n-butyl, isobutyl, tert-butyl or pentyl groups. For example, R₁ and R₂ may both be ethyl groups.

The radical polymerization initiator can be chosen from any conventional polymerization initiator, such as azo compounds, for example, azobisisobutyronitrile; peroxide compounds, such as organic peroxides comprising from 6 to 30 carbon atoms, such as benzoyl peroxide.

For example, a nitroxide/initiator molar ratio ranging, for example, from 1:1 to 2.5:1 is observed; this ratio can range, further, for example, from 2:1 to 2.5:1 when it is considered that one mole of initiator gives rise to two moles of polymer chains and can range from 1:1 to 1.25:1 for monofunctional initiators.

In another embodiment, it is possible to employ, as the radical polymerization initiator, alkoxyamines of formula (II)

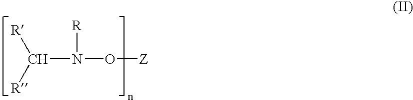

wherein:

R, R′ and R″, which may be identical or different, have the meanings given above, for the nitroxide of formula (I),

n is an integer of less than or equal to 8, for example, ranging from 1 to 3; and

Z is chosen from monovalent and polyvalent radicals, for example, from styryl, acryloyl and methacryloyl radicals,

which can advantageously be chosen in order to initiate the polymerization and, at the same time, release the nitroxide which controls this polymerization.

A nitroxide of formula (I) can also be added to the alkoxyamine of formula (II) in a proportion ranging, for example, from 0 to 20 mol % with respect to the moles of alkoxyamine functional groups (one mole of polyvalent alkoxyamine contributes a number of alkoxyamine functional groups proportional to its valency), so as to improve the quality of the polymerization control.

A person skilled in the art will know how to choose the initiator according to the requirements of the composition disclosed herein. Thus, a monofunctional initiator will result in asymmetric chains, whereas a polyfunctional initiator will result in macromolecules having a symmetry starting from a core.

The at least one film-forming gradient copolymer disclosed herein can be present in the cosmetic varnish composition disclosed herein in an amount ranging, for example, from 0.1 to 60% by weight, relative to the total weight of the composition, further, for example, ranging from 0.2 to 40% by weight, relative to the total weight of the composition, and, further, for example, ranging from 1 to 35% by weight, relative to the total weight of the composition, and, even further, for example, from 5 to 30% by weight, relative to the total weight of the composition.

The at least one film-forming gradient copolymer can be present in the composition disclosed herein in the dissolved form, for example, dissolved in water or an organic solvent, or else in the form of an aqueous or organic dispersion.

A polymer is “soluble” when it forms, at 1% by weight, a clear solution at 25° C.

It is “dispersible” when it forms, at 1% by weight, at 25° C., a stable suspension of fine particles, generally spherical particles, the mean size of which is, for example, less than 1 micron, for example, ranging from 5 to 400 nm, further, for example, from 10 to 250 nm, measured by light scattering.

It is possible to prepare an aqueous or organic solution or dispersion of the copolymer by directly mixing the polymer with water or the organic solvent, optionally while heating.

When the at least one film-forming gradient copolymer comprises hydrophilic units, for example, an aqueous solution or dispersion can be prepared by dissolving the copolymer in an organic solvent with a lower boiling point than water (for example, acetone or methyl ethyl ketone), at a level of solid ranging from 20 to 90% by weight.

When the hydrophilic monomers are acid monomers, a solution, for example, at least 1M, of base, such as a hydroxonium ion (OH⁻) salt, an amine (ammonia), a carbonate (CO₃²⁻) salt or a hydrogencarbonate (HCO₃⁻) salt, or of organic neutralizing agent can be added to the organic solution. When the hydrophilic monomers are amine monomers, a solution, for example, at least 1M, of acid can be added. Water is then added to the solution with vigorous stirring in a proportion such that the level of solid obtained ranges from 1 to 80% by weight. The water can optionally be replaced by an aqueous/alcoholic mixture in proportions ranging from 99/1 to 50/50. The solvent is evaporated while stirring the solution at 100° C. Concentration is continued until the desired level of solid is obtained.

The adhesion of the nail varnish film is determined using the right-angle lattice pattern test defined in Standard ASTM D3359-7.

A layer with a thickness of 50 μm, after drying at 23±2° C. and at 55±5% relative humidity for 24 hours, is deposited on a polyamide sheet sold under the name Nylon 6 by Goodfellow exhibiting a roughness ranging from 10 to 500 nm. The film is cut in the form of a right-angle lattice pattern in accordance with Standard ASTM D3359 using a comb with 6 blades each spaced 1 mm apart. The film is thus cut throughout its thickness. A standardized adhesive paper (ISO 2409) is then applied to the cut film so that the adhesive paper is in contact with the entire surface of the cut film. The adhesive paper is then removed from the film with the right-angle lattice pattern and the number of squares of film which have been detached from the sheet by the adhesive paper is determined. The detachment percentage of the film is then determined as described in Standard ASTM D3359, for a mean of 10 samples.

The varnish film disclosed herein exhibits a detachment percentage, for example, of less than 45%, further, for example, of less than 40% and, even further, for example, of less than 35%.

Moreover, in one embodiment, the varnish composition disclosed herein is capable of forming a film having a damping power “tgδ” ranging from 0.5 to 1.6, further, for example, ranging from 0.8 to 1.4.

For example, the varnish composition disclosed herein is also capable of forming a film having a storage modulus E′, for example, of less than 300 MPa, further, for example, of less than 100 MPa and, even further, for example, of less than 80 MPa.

The storage modulus E′ and the damping power are determined according to the protocol of viscoelastometry tests carried out by DMTA (Dynamical and Mechanical Temperature Analysis) with a DMTA device from TA Instruments (DMA2980 model) on a sample of polymer film of 150±50 μm in thickness, 5 mm in width and 10 mm in length, after drying at 23° C. and at a relative humidity ranging from 50 to 55% for 24 hours.

A tensile stress can be imposed on this sample. The sample is subjected to a static force of 0.01N, on which a sinusoidal displacement of ±8 μm is superimposed at a frequency of 20 Hz. The operation is thus carried out in the linear region, under low levels of deformation. This tensile stress is applied to the sample at temperatures ranging from −150° C. to +220° C., with a variation in temperature of 3° C. per minute.

The complex modulus E*=E′+iE″ of the polymer tested is then measured as a function of the temperature.

From these measurements, the dynamic storage modulus E′ and the dynamic loss modulus E″ are deduced, as well as the damping power: tgδ=E″/E′.

The curve of the values of tgδ as a function of the temperature is then plotted; this curve exhibits at least one peak. The glass transition temperature Tg of the polymer corresponds to the temperature at which the top of this peak is situated.

When the curve exhibits at least 2 peaks (in this case, the polymer exhibits at least 2 Tg), the Tg value of the polymer tested is taken as being the temperature for which the curve exhibits the peak with the greater amplitude (that is to say, corresponding to the higher value of tgδ; in this case, only the “majority” Tg is regarded as Tg value of the polymer tested).

The at least one film-forming gradient copolymer may, for example, be insoluble in water at 25° C., that is to say that it is soluble to less than 1% by weight in water at 25° C. (solubility of less than 1% by weight). The at least one film-forming gradient copolymer may, for example, be soluble in organic solvents, such as ethyl acetate, butyl acetate or methyl acetate (solubility of greater than 90% by weight at 25° C.).

The cosmetic compositions disclosed herein comprise, in addition to the at least one film-forming gradient copolymer, a cosmetically acceptable medium, that is to say a medium compatible with nails.

The composition can thus furthermore comprise at least one additional film-forming polymer chosen, for example, from radical synthetic polymers of polycondensate synthetic polymers, polymers of natural origin and blends thereof, such as acrylic polymers, polyurethanes, polyesters, polyamides, polyureas and cellulose polymers, such as nitrocellulose. The at least one additional film-forming polymer may also, for example, be chosen from resins, such as sulphonamide resins, alkyd resins and cellulose esters, such as cellulose acetate/butyrate, cellulose acetate or cellulose acetate/propionate.

The at least one additional film-forming polymer may be present in an amount ranging, for example, from 0.01 to 50% by weight, relative to the total weight of the composition, and further, for example, from 1 to 30% by weight, relative to the total weight of the composition.

The nail varnish composition disclosed herein can also comprise at least one additional agent which is able to form a film for improving the film-forming properties of the varnish. The at least one additional agent which is able to form a film can be chosen from any compounds known to a person skilled in the art as being capable of fulfilling the desired role and can be chosen, for example, from plasticizers.

For example, the plasticizers may be chosen from at least one of:

citrates, such as triethyl citrate, tributyl citrate, triethyl acetylcitrate, tributyl acetylcitrate and tri(2-ethylhexyl) acetylcitrate;

phthalates, such as diethyl phthalate, dibutyl phthalate, dioctyl phthalate, dipentyl phthalate and dimethoxyethyl phthalate; and

tricresyl phosphate, benzyl benzoate, tributyl phosphate, butyl acetylricinoleate, glyceryl acetylricinoleate, butyl glycolate, tributoxyethyl phosphate, triphenyl phosphate, dibutyl tartrate, camphor, glyceryl triacetate and N-ethyl-o,p-toluenesulphonamide.

The plasticizer can be present in the composition disclosed herein in an amount ranging, for example, from 0.01 to 10% by weight, relative to the total weight of the composition, and, further, for example, from 0.1 to 5% by weight, relative to the total weight of the composition. The plasticizer may, for example, be present in the composition according to a at least one film-forming gradient film-forming polymer/plasticizer ratio by weight ranging from 1.5:1 to 3:1.

The composition disclosed herein can comprise a medium chosen from an aqueous medium, an aqueous/organic medium and an organic solvent medium. In one embodiment, the medium is an an organic solvent medium. It can, further, for example, be an anhydrous medium.

The aqueous medium of the composition can comprise essentially water. For example, the content of water in the composition can range, for example, from 10% to 95% by weight, relative to the total weight of the composition, and further, for example, from 40% to 90% by weight, relative to the total weight of the composition, and, even further, for example, from 60% to 85% by weight, relative to the total weight of the composition.

When the composition comprises an aqueous medium, the at least one film-forming gradient copolymer can be present therein in the form of solid particles dispersed in the aqueous medium or in a dissolved form.

The composition disclosed herein can also comprise at least one organic solvent, for example, at least one organic solvent chosen from water-miscible organic solvents, such as monoalcohols comprising from 1 to 5 carbon atoms, glycols comprising from 2 to 8 carbon atoms, C₃-C₄ ketones and C₂-C₄ aldehydes, for example, in a content which can range from 0.1% to 15% by weight, relative to the total weight of the composition.

The organic solvent medium or aqueous/organic medium of the composition disclosed herein can comprise, for example, at least one organic solvent chosen from:

ketones which are liquid at ambient temperature, such as methyl ethyl ketone, methyl isobutyl ketone, diisobutyl ketone, isophorone, cyclohexanone and acetone;

alcohols which are liquid at ambient temperature, such as ethanol, isopropanol, n-propanol, n-butanol, diacetone alcohol, 2-butoxyethanol and cyclohexanol;

glycols which are liquid at ambient temperature, such as ethylene glycol, propylene glycol, pentylene glycol and glycerol;

propylene glycol ethers which are liquid at ambient temperature, such as propylene glycol monomethyl ether, propylene glycol monomethyl ether acetate and dipropylene glycol mono(n-butyl) ether;

short-chain esters (comprising from 3 to 8 carbon atoms in total), such as ethyl acetate, methyl acetate, propyl acetate, n-butyl acetate and isopentyl acetate;

ethers which are liquid at ambient temperature, such as diethyl ether, dimethyl ether and dichlorodiethyl ether;

alkanes which are liquid at ambient temperature, such as decane, heptane, dodecane, isododecane and cyclohexane;

aromatic cyclic compounds which are liquid at ambient temperature, such as toluene and xylene; and

aldehydes which are liquid at ambient temperature, such as benzaldehyde and acetaldehyde.

The at least one organic solvent can be present in the composition disclosed herein in an amount ranging, for example, from 10% to 95% by weight, relative to the total weight of the composition, further, for example, from 40% to 90% by weight, relative to the total weight of the composition, and, even further, for example, from 60% to 85% by weight, relative to the total weight of the composition.

The composition can further comprise at least one thickening agent, for example, for conferring a consistency on the composition which may make possible good application of the composition to the nails.

The at least one thickening agent can, for example, be chosen from thickeners of organic solvents and can, further, for example, be chosen from hydrophobic silicas, such as those disclosed in document EP-A-898 960 and, for example, sold under the references “AEROSIL R812” by Degussa, “CAB-O-SIL TS-530”, “CAB-O-SIL TS-610” or “CAB-O-SIL TS-720” by CABOT or “AEROSIL R972” or “AEROSIL R974” by Degussa; clays, such as montmorillonite, stearalkonium hectorite and stearalkonium bentonite; and polysaccharide alkyl ethers (for example, wherein the alkyl group comprises from 1 to 24 carbon atoms, further, for example, from 1 to 10 carbon atoms, further, for example, from 1 to 6 carbon atoms, and, even further, for example, from 1 to 3 carbon atoms), such as those disclosed in the document EP-A-898 958 and, for example, sold under the names “N-HANCE-AG 200” and “N-HANCE AG 50” by Aqualon.

The at least one thickening agent can be present in the composition disclosed herein in an amount ranging, for example, from 0.05% to 10% by weight, relative to the total weight of the composition and further, for example, ranging from 0.1% to 3% by weight, relative to the total weight of the composition.

The composition disclosed herein can further comprise at least one coloring material chosen from water-soluble dyes, fat-soluble dyes and pulverulent coloring materials, such as pigments, pearlescence agents and glitters well known to a person skilled in the art.

The at least one coloring material can be present in the composition disclosed herein in an amount ranging, for example, from 0.01 to 50% by weight, relative to the total weight of the composition and, further, for example, from 0.05 to 30% by weight, relative to the total weight of the composition.

As used herein, the term “pigments” should be understood as meaning white or colored and inorganic or organic particles of any shape which are insoluble in the physiological medium and which are intended to color the composition. The term “pearlescence agents” should be understood as meaning iridescent particles of any shape, for example, produced by certain molluscs in their shells or else synthesized.

The pigments can be white or colored and inorganic and/or organic. For example, the pigments may be chosen from inorganic pigments, of titanium dioxide, optionally surface treated, zirconium or cerium oxides, zinc, iron or chromium oxides (the iron oxides being black, yellow or red), manganese violet, ultramarine blue, chromium hydrate, and ferric blue or metal powders, such as aluminium powder or copper powder.

For example, the organic pigments may be chosen from carbon black, D & C pigments, and lakes based on cochineal carmine or barium, strontium, calcium or aluminium.

The pearlescent pigments can, for example, be chosen from white pearlescent pigments, such as mica covered with titanium oxide or with bismuth oxychloride, colored pearlescent pigments, such as titanium oxide-coated mica covered with iron oxides, titanium oxide-coated mica covered with, for example, ferric blue or with chromium oxide, or titanium oxide-coated mica covered with an organic pigment of the abovementioned type, and pearlescent pigments based on bismuth oxychloride.

For example, the water-soluble dyes may be chosen from the disodium salt of ponceau, the disodium salt of alizarin green, the trisodium salt of amaranth, the disodium salt of tartrazine, the monosodium salt of rhodamine, the disodium salt of fuchsin, xanthophyll and methylene blue.

The fat-soluble dyes can, for example, be chosen from Sudan red, DC Red 17, DC Green 6, β-carotene, soybean oil, Sudan brown, DC Yellow 11, DC Violet 2, DC Orange 5 and quinoline yellow.

The dyes can be present in the composition disclosed herein in an amount ranging, for example, from 0.01% to 6% by weight, relative to the total weight of the composition, and further, for example, from 0.05% to 3% by weight, relative to the total weight of the composition.

The pigments, pearlescence agents and glitters can be present in the composition disclosed herein, for example, in the base and/or surface composition, in an amount ranging, for example, from 0.01% to 25% by weight, relative to the total weight of the composition, and further, for example, from 0.05% to 15% by weight, relative to the total weight of the composition.

The composition disclosed herein can also comprise at least one constituent chosen from those commonly used in cosmetics, such as fillers, spreading agents, wetting agents, dispersing agents, antifoaming agents, preservatives, UV screening agents, active principles, surfactants, moisturizing agents, fragrances, stabilizing agents, antioxidants, vitamins, trace elements, basifying agents, acidifying agents, and ceramides. Of course, a person skilled in the art will take care to choose this or these optional additional compounds, and/or their amounts, so that the advantageous properties of the composition disclosed herein are not, or not substantially, detrimentally affected by the envisaged addition(s).

The nail varnish composition can be provided in a form, for example, chosen from bases for varnishes; products for making up the nails; finishing compositions, also known as top coat, to be applied to a product for making up the nails, and/or as a product for the cosmetic care of the nails. These compositions can be applied to the nails of human beings or else to false nails.

Further disclosed herein is a cosmetic process for making up and/or caring for the nails, comprising applying to the nails the cosmetic composition defined herein.

The invention is illustrated in more detail in the following examples, without, however, being limiting in nature.

In these examples, the stable nitroxide, known as SG1, of formula:

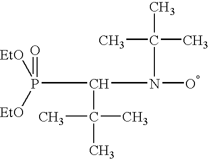

is used as agent for controlling the polymerization.

The polymerization initiators mentioned in the examples are alkoxyamines known as “DIAMS” and “MONAMS” which correspond to the following formulae:

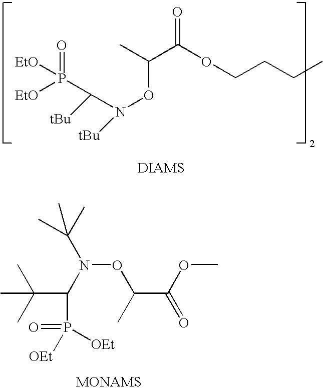

### EXAMPLE 1

### Bulk Synthesis of Gradient Copolymer

The mixture of reactants was as follows:

| | |
|---|---|
| MONAMS: 3.0 g | |
| SG1: 0.18 g | |
| Ethyl acrylate: 480 g | i.e. 80% by weight/total weight of monomers |
| Styrene: 60 g | i.e. 10% by weight/total weight of monomers |
| Methacrylic acid: 60 g | i.e. 10% by weight/total weight of monomers |

The combined constituents were mixed, in the absence of solvent, under a nitrogen atmosphere and were then heated at a temperature ranging from 110 to 115° C. for 198 minutes. The reaction was stopped at a degree of conversion of 60%.

Simulated calculation of the gradient gave the curve below. The theoretical prediction gave 30% incorporation of the (styrene/methacrylic acid) mixture and 70% ethyl acrylate.

This model was validated by monitoring the relative concentrations of the three monomers by gas chromatography and NMR analysis of the polymers.

By these methods, it was found that, at 60% conversion, the final chemical composition of the copolymer was as follows (% by weight): 68.4% ethyl acrylate, 16.1% styrene and 15.5% methacrylic acid according to NMR spectroscopy on the calculated curve (69%).

[image]

By LAC, the plot of the polymer showed the low polydispersity of the chemical composition of the chains.

Measurement of the masses by steric exclusion chromatography gave the following results:

Mn=32 140 g/mol and Mw=51 700 g/mol, resulting in a polydispersity index PI=1.6. The dispersity in composition (or w) was 1.6.

A diagrammatic representation of the copolymer obtained may be as follows:

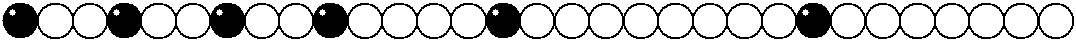

in which the dark spheres are styrene/methacrylic acid sequences and the white spheres are ethyl acrylate sequences.

### EXAMPLE 2

### Bulk Synthesis of Gradient Copolymer

Various copolymers were prepared according to the procedure described in Example 1, starting from the following mixture of reactants:

MONAMS: 3.0 g

SG1: 0.18 g

Styrene: 60 g

Methacrylic acid: 60 g

Acrylate (or mixture of acrylate): 480 g

| | | | Final composition of |
|---|---|---|---|
| | | Characteristics of | the copolymer (% by |
| Example | Acrylate | the copolymer | weight) |
| 2a | Butyl acrylate | Mn = 31 100 g/mol | Styrene: 18 |
| | | Mw = 52 930 g/mol | Methacrylic Ac.: 22 |
| | | Pl = 1.7 | Butyl acrylate: 60 |
| 2b | Methyl acrylate | Mn = 32 750 g/mol | Styrene: 20 |
| | | Mw = 61 470 g/mol | Methacrylic Ac.: 21 |
| | | Pl = 1.88 | Methyl acrylate: 59 |
| 2c | 50/50 Butyl | Mn = 29 690 g/mol | Styrene: 18 |
| | acrylate/ethyl | Mw = 51 630 g/mol | Methacrylic Ac.: 16 |
| | acrylate | Pl = 1.74 | Acrylates: 66 |
| | mixture | | |
| | (by weight) | | |

### EXAMPLE 3

### Synthesis in the Presence of Solvent

The same synthesis as in Example 1 was carried out but in the presence of solvent.

The mixture of reactants was as follows:

MONAMS: 3.43 g

SG1: 0.2 g

Ethyl acrylate: 336 g

Styrene: 42 g

Methacrylic acid: 42 g

Toluene: 180 g

The combined constituents were mixed, in toluene as solvent, under a nitrogen atmosphere and were then heated at a temperature ranging from 110 and 115° C. for 198 minutes.

The final degree of conversion was 82% and the level of solid obtained was 57.2% by weight.

The following analytical results were determined:

Mn=30 570 g/mol, Mw=50 500 g/mol and PI=1.65.

The dispersity in composition (or w) was 2.0.

The final composition of the copolymer was given by liquid adsorption chromatography (LAC), which indicated the similarity in composition with the copolymer prepared in Example 1 and the absence of homopolymer in the materials.

### EXAMPLE 4

### Synthesis in the Presence of Solvent

The synthesis of a new copolymer was carried out according to the process of Example 3, at 120° C. and for 400 minutes, but in a different solvent: methyl ethyl ketone.

The starting composition of the mixture was:

MONAMS: 4.893 g

SG1: 0.2881 g

Ethyl acrylate: 293.8 g

Methyl acrylate: 32.66 g

Styrene: 76.8 g

Methacrylic acid: 76.8 g

Methyl ethyl ketone: 120 g

The final degree of conversion was 99% and the level of solid obtained was 79.9%.

The following analytical results were determined:

Mn=30 500 g/mol

Mw=58 000 g/mol

PI=1.9

The incorporation of the monomers over time was measured by monitoring, by gas chromatography, the levels of residual monomers (in %) over time (in minutes):

| | | time | | | | | |
|---|---|---|---|---|---|---|---|
| | | 0 | 75 | 130 | 190 | 290 | 400 |
| | | Overall conversion | | | | | |
| | | 0 | 16 | 30.5 | 49.5 | 85.4 | 99 |
| Residual monomers | MeA | 5.45 | 5.1 | 3.75 | 3.75 | 1.6 | 0.13 |
| (%) | EA | 48.95 | | | | 17.95 | 1.2 |
| | MAA | 12.8 | 12.15 | 4.6 | 2 | 0.35 | 0.08 |
| | S | 12.8 | 12.46 | 6.7 | 3.92 | 0.15 | 0.007 |

Ethyl acrylate: EA
Methyl acrylate: MeA
Styrene: S
Methacrylic acid: MAA

The total level of residual monomer was calculated taking into account the quantified by the level of solid.

It was noted that each monomer was present throughout the reaction. The determined for each monomer was then be calculated and gave the following curves:

[image]

The final composition of the copolymer was as follows:

ethyl acrylate: 34% by weight

methyl acrylate: 34% by weight

styrene: 16% by weight

methacrylic acid: 16% by weight

### EXAMPLE 5

The copolymers of Examples 1, 2a and 2c were dissolved in butyl acetate, so as to obtain a solution having a dry matter content of 10% by weight. The solution obtained was subsequently applied to the nails. After drying, a varnish film was obtained which had the following characteristics, measured according to the protocols described above in the description:

| | Damping | | Storage modulus |
|---|---|---|---|
| | power (tgδ) | Detachment (%) | (E′ in MPa) |
| Example 1 | 1.1 | Less than 30% | 50 MPa |
| Example 2a | 1.1 | Less than 30% | 60 MPa |
| Example 2c | 1.0 | Less than 30% | 55 MPa |

### EXAMPLE 6

A nail varnish composition was prepared which had the following composition:

| | |
|---|---|
| Polymer of Example 1 | 23.8 g of active material |
| Butyl acetate | 25.0 g |
| Isopropanol | 10.7 g |
| Hexylene glycol | 2.5 g |
| Pigment (DC Red 7 Lake) | 1 g |
| Modified hectorite | 1.3 g |
| (Bentone ® 27 V from Elementis) | |

After application to the nails, this varnish was judged to exhibit very good properties of hold and of impact strength.

What is claimed is:

1. A nail varnish composition comprising, in a cosmetically acceptable medium, at least one film-forming gradient copolymer comprising at least two different monomeric units and exhibiting a mass polydispersity index (PI) of less than or equal to 2.5, wherein the composition is capable of forming a film having an adhesion, measured according to Standard ASTM D 3359-7, corresponding to a detachment percentage of less than 45%.

2. The composition according to claim 1, wherein the mass polydispersity index (PI) ranges from 1.1 to 2.3.

3. The composition according to claim 2, wherein the mass polydispersity index (PI) ranges from 1.15 to 2.0.

4. The composition according to claim 3, wherein the mass polydispersity index (PI) ranges from 1.2 to 1.9.

5. The composition according to claim 4, wherein the mass polydispersity index (PI) ranges from 1.2 to 1.8.

6. The composition according to claim 1, wherein the detachment percentage is less than 40%.

7. The composition according to claim 6, wherein the detachment percentage is less than 35%.

8. The composition according to claim 1, wherein the weight-average molecular mass of the at least one film-forming gradient copolymer ranges from 5 000 g/mol to 1 000 000 g/mol.

9. The composition according to claim 8, wherein the weight-average molecular mass of the at least one film-forming gradient polymer ranges from 5 500 g/mol to 800 000 g/mol.

10. The composition according to claim 9, wherein the weight-average molecular mass of the at least one film-forming gradient polymer ranges from 6 000 g/mol to 500 000 g/mol.

11. The composition according to claim 1, wherein the number-average molecular mass of the at least one film-forming gradient copolymer ranges from 5 000 g/mol to 1 000 000 g/mol.

12. The composition according to claim 11, wherein the number-average molecular mass of the at least one film-forming gradient polymer ranges from 5 500 g/mol to 800 000 g/mol.

13. The composition according to claim 12, wherein the number-average molecular mass of the at least one film-forming gradient polymer ranges from 6 000 g/mol to 500 000 g/mol.

14. The composition according to claim 1, wherein the at least one film-forming gradient copolymer comprises polymer chains each comprising at least one monomeric unit Mi, such that there is a nonzero probability of encountering the monomeric unit Mi along each chain, regardless of the normalized position (x) on the polymer chain.

15. The composition according to claim 1, wherein the at least one film-forming gradient copolymer is such that, on the adsorption chromatography (“LAC”) curve representing the proportion of polymers as a function of the elution volume, the difference (V^{1/2} max−V^{1/2} min) is less than or equal to 3.5, wherein V^{1/2} min is the minimum value of the elution volume at mid-height of the curve and V^{1/2} max is the maximum value of the elution volume at mid-height of the curve.

16. The composition according to claim 15, wherein the difference (V^{1/2} max−V^{1/2} min) ranges from 1 to 2.8.

17. The composition according to claim 16, wherein the difference (V^{1/2} max−V^{1/2} min) ranges from 1.2 to 2.5.

18. The composition according to claim 1, wherein the at least one film-forming gradient copolymer comprises at least two different monomeric units which are each present in an amount ranging from 1 to 99% by weight, relative to the total weight of the final copolymer.

19. The composition according to claim 18, wherein the at least one film-forming gradient copolymer comprises at least two different monomeric units which are each present in an amount ranging from 2 to 98% by weight, relative to the total weight of the final copolymer.

20. The composition according to claim 19, wherein the at least one film-forming gradient copolymer comprises at least two different monomeric units which are each present in an amount ranging from 5 to 95% by weight, relative to the total weight of the final copolymer.

21. The composition according to claim 1, wherein the at least one film-forming gradient copolymer comprises at least one hydrophilic monomeric unit which is present in an amount ranging from 1 to 99% by weight, relative to the total weight of the copolymer.

22. The composition according to claim 21, wherein the at least one film-forming gradient copolymer comprises at least one hydrophilic monomeric unit which is present in an amount ranging from 2 to 70% by weight, relative to the total weight of the copolymer.

23. The composition according to claim 22, wherein the at least one film-forming gradient copolymer comprises at least one hydrophilic monomeric unit which is present in an amount ranging from 3 to 50% by weight, relative to the total weight of the copolymer.

24. The composition according to claim 23, wherein the at least one film-forming gradient copolymer comprises at least one hydrophilic monomeric unit which is present in an amount ranging from 4 to 30% by weight, relative to the total weight of the copolymer.

25. The composition according to claim 24, wherein the at least one film-forming gradient copolymer comprises at least one hydrophilic monomeric unit which is present in an amount ranging from 5 to 25% by weight, relative to the total weight of the copolymer.

26. The composition according to claim 1, wherein the at least one film-forming gradient copolymer comprises at least one monomeric unit, the homopolymer of which has a Tg of less than or equal to 20° C.

27. The composition according to claim 26, wherein the homopolymer has a Tg ranging from −150° C. to 20° C.

28. The composition according to claim 27, wherein the homopolymer has a Tg ranging from −130° C. to 18° C.

29. The composition according to claim 28, wherein the homopolymer that has a Tg ranging from −120° C. to 15° C.

30. The composition according to claim 26, wherein the at least one monomeric unit, the homopolymer of which has a Tg≦20° C., is present in an amount of monomeric unit ranging from 1 to 99% by weight, relative to the total weight of the copolymer.

31. The composition according to claim 30, wherein the at least one monomeric unit, the homopolymer of which has a Tg≦20° C., is present in an amount of monomeric unit ranging from 20 to 90% by weight, relative to the total weight of the copolymer.

32. The composition according to claim 31, wherein the at least one monomeric unit, the homopolymer of which has a Tg≦20° C., is present in an amount of monomeric unit ranging from 30 to 85% by weight, relative to the total weight of the copolymer.

33. The composition according to claim 32, wherein the at least one monomeric unit, the homopolymer of which has a Tg≦20° C., is present in an amount of monomeric unit ranging from 50 to 75% by weight, relative to the total weight of the copolymer.

34. The composition according to claim 1, wherein the at least one film-forming gradient copolymer comprises at least one hydrophilic monomeric unit chosen from units of:
amino(C₁-C₄ alkyl) (meth)acrylate derivatives;
N,N-di(C₁-C₄ alkyl)amino(C₁-C₆ alkyl)(meth)acrylamides;
di(C₁-C₈ alkyl)allylamines;
vinylamine;
vinylpyridines;
and the salts thereof with inorganic acids or organic acids and the quaternized forms thereof of all the abovementioned compounds;
ethylenic carboxylic acids;
carboxylic anhydrides comprising at least one vinyl bond;
ethylenic sulphonic acids and the salts thereof;
the potassium salt of 3-(acryloyloxy)propanesulphonic acid and the compound of formula CH₂═CHCOOCH₂OCH₂(OH)CH₂SO₃⁻Na⁺;
amides of unsaturated carboxylic acids;
hydroxyalkyl (meth)acrylates;
(meth)acrylates of polyethylene glycol (5 to 100 EO) and of glycol which are optionally substituted on their end functional group by at least one group chosen from alkyl, phosphate, phosphonate and sulphonate groups;
alkoxyalkyl (meth)acrylates;
polysaccharide (meth)acrylates;
vinylamides;
vinyl ethers;
methacrylamidopropoxytrimethylammonium betaine;
N,N-dimethyl-N-methacryloyloxyethyl-N-(3-sulphopropyl)ammonium betaine;
3-methacryloylethoxycarbonylpyridinium;
the compound of formula: 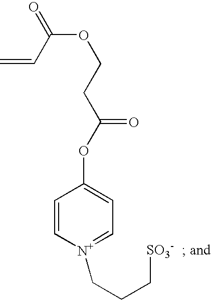
4-vinylpyridiniumsulphopropyl betaine of formula:

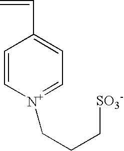

35. The composition according to claim 34, wherein the amino(C₁-C₄ alkyl) (meth)acrylate derivatives are chosen from N,N-di(C₁-C₄ alkyl)amino(C₁-C₆ alkyl) (meth)acrylates.

36. The composition according to claim 35, wherein the N,N-di(C₁-C₄ alkyl)amino(C₁-C₆ alkyl) (meth)acrylates are chosen from N,N-dimethylaminoethyl methacrylate (MADAME) and N,N-diethylaminoethyl methacrylate (DEAMEA).

37. The composition according to claim 34, wherein the N,N-di(C₁-C₄ alkyl)amino(C₁-C₆ alkyl)(meth)acrylamides are chosen from N,N-dimethylacrylamide, N,N-dimethylaminopropylacrylamide (DMAPA) and N,N-dimethylaminopropylmethacrylamide (DMAPMA).

38. The composition according to claim 34, wherein the di(C₁-C₈ alkyl)allylamines are dimethyldiallylamines.

39. The composition according to claim 34, wherein the vinylpyridines are chosen from 2-vinylpyridine and 4-vinylpyridine.

40. The composition according to claim 34, wherein the ethylenic carboxylic acids are chosen from monocarboxylic acid and dicarboxylic acids.

41. The composition according to claim 40, wherein the monocarboxylic acid and dicarboxylic acids are chosen from acrylic acid, methacrylic acid, crotonic acid, itaconic acid, fumaric acid and maleic acid.

42. The composition according to claim 34, wherein the carboxylic anhydrides comprising at least one vinyl bond are maleic anhydrides.

43. The composition according to claim 34, wherein the ethylenic sulphonic acids are chosen from styrenesulphonic acid, acrylamidopropanesulphonic acid, and the salts thereof.

44. The composition according to claim 34, wherein the amides of unsaturated carboxylic acids are chosen from acrylamide and methacrylamide and the N-substituted derivatives thereof.

45. The composition according to claim 34, wherein the amides of unsaturated carboxylic acids are chosen from N-(C₁-C₄ alkyl)(meth)acrylamides and N,N-di(C₁-C₄ alkyl)(meth)acrylamides.

46. The composition according to claim 45, wherein the N-(C₁-C₄ alkyl)(meth)acrylamides are N-methylacrylamides.

47. The composition according to claim 46, wherein the N,N-di(C₁-C₄ alkyl) (meth)acrylamides are N,N-dimethylacrylamides.

48. The composition according to claim 47, wherein the hydroxyalkyl (meth)acrylates are chosen from hydroxyalkyl (meth)acrylates comprising an alkyl group comprising from 2 to 4 carbon atoms.

49. The composition according to claim 48, wherein the hydroxyalkyl (meth)acrylates are hydroxyethyl (meth)acrylates.

50. The composition according to claim 34, wherein the (meth)acrylates of polyethylene glycol (5 to 100 EO) and of glycol which are optionally substituted on their end functional group by at least one group chosen from alkyl, phosphate, phosphonate and sulphonate groups are chosen from glyceryl acrylate, methoxypolyethylene glycol (8 or 12 EO) (meth)acrylate and hydroxypolyethylene glycol (meth)acrylate.

51. The composition according to claim 34, wherein the alkoxyalkyl (meth)acrylates are ethoxyethyl (meth)acrylates.

52. The composition according to claim 34, wherein the polysaccharide (meth)acrylates are sucrose acrylates.

53. The composition according to claim 34, wherein the vinylamides are chosen from vinylacetamide and optionally cyclic vinylamides.

54. The composition according to claim 53, wherein the cyclic vinylamides are chosen from vinyllactams.

55. The composition according to claim 54, wherein the vinyllactams are chosen from N-vinylpyrrolidone and N-vinylcaprolactam.

56. The composition according to claim 34, wherein the vinyl ethers are vinyl methyl ethers.

57. The composition according to claim 1, wherein the at least one film-forming gradient copolymer comprises at least one hydrophilic monomeric unit chosen from units of N,N-dimethylaminoethyl methacrylate (MADAME), acrylic acid, methacrylic acid, crotonic acid, styrenesulphonic acid, acrylamidopropanesulphonic acid, dimethylaminopropylmethacrylamide (DMAPMA), styrenesulphonate, hydroxyethyl acrylate, glyceryl acrylate, ethoxyethyl methacrylate, ethoxyethyl acrylate, methoxypolyethylene glycol (8 or 12 EO) (meth)acrylate, hydroxypolyethylene glycol (meth)acrylate, N-vinylpyrrolidone, N-vinylcaprolactam, acrylamide and N,N-dimethylacrylamide.

58. The composition according to claim 1, wherein the at least one film-forming gradient copolymer comprises at least one monomeric unit chosen from units of C₁-C₄ alkyl (meth)acrylates, the C₁-C₄ alkyl (meth)acrylates resulting in the production of (meth)acrylic acid after hydrolysis.

59. The composition according to claim 58, wherein the C₁-C₄ alkyl (meth)acrylates are chosen from tert-butyl (meth)acrylate and ethyl (meth)acrylate.

60. The composition according to claim 1, wherein the at least one film-forming gradient copolymer comprises at least one monomeric unit, the homopolymer of which has a Tg of less than or equal to 20° C., wherein the at least one monomeric unit is chosen from units of:
ethylenic hydrocarbons comprising from 2 to 10 carbons;
acrylates of formula CH₂═CHCOOR₁, wherein R₁ is chosen from saturated and unsaturated, linear and branched, hydrocarbonaceous groups comprising from 1 to 12 carbons with the exception of the tert-butyl group, wherein at least one heteroatom chosen from O, N, S and Si is optionally inserted, it being possible in addition for the hydrocarbonaceous groups to be optionally substituted with at least one substituent chosen from hydroxyl groups and from halogen atoms chosen from Cl, Br, I and F atoms; or alternatively R₁ is an —(R″)ₓ—(OC₂H₄)ₙ—OR′″ group, wherein x=0 or 1, R″ is chosen from saturated and unsaturated, linear and branched, hydrocarbonaceous groups comprising from 1 to 12 carbons, n is a number ranging from 5 to 100 and R′″ is chosen from H and CH₃;
methacrylates of formula: CH₂═C(CH₃)—COOR₂, wherein R₂ is chosen from saturated and unsaturated, linear and branched, hydrocarbonaceous groups comprising from 3 to 12 carbon atoms wherein at least one heteroatom chosen from O, N, S and Si is optionally inserted, it being possible in addition for the hydrocarbonaceous groups to be optionally substituted with at least one substituent chosen from hydroxyl groups and halogen atoms chosen from Cl, Br, I and F atoms; or alternatively R₂ is an —(R″)ₓ—(OC₂H₄)ₙ—OR′″ group, wherein x=0 or 1, R″ is chosen from saturated and unsaturated, linear and branched, hydrocarbonaceous groups comprising 1 to 12 carbons, n is a number ranging from 5 to 100 and R′″ is chosen from H and CH₃; —N— or N,N-substituted derivatives of unsaturated C₁₋₁₂ carboxylic acid amides;
vinyl esters of formula: R₃—CO—O—CH═CH₂, wherein R₃ is chosen from linear and branched alkyl groups comprising from 2 to 12 carbons; and
vinyl alkyl ethers, wherein the alkyl group comprises from 1 to 12 carbons,

61. The composition according to claim 60, wherein the ethylenic hydrocarbons comprising from 2 to 10 carbons are chosen from ethylene, isoprene and butadiene.

62. The composition according to claim 60, wherein the N- or N,N-substituted derivatives of unsaturated C₁₋₁₂ carboxylic acid amides are chosen from N-(C₁₋₁₂ alkyl) (meth)acrylamides.

63. The composition according to claim 62, wherein the N-(C₁₋₁₂ alkyl)(meth)acrylamides are N-octylacrylamides.

64. The composition according to claim 60, wherein, in formula R₃—CO—O—CH═CH₂, the alkyl groups of R₃ are chosen from vinyl propionate, vinyl butyrate, vinyl ethylhexanoate, vinyl neononanoate and vinyl neododecanoate.

65. The composition according to claim 60, wherein the vinyl alkyl ethers are chosen from methyl vinyl ether and ethyl vinyl ether.

66. The composition according to claim 1, wherein the at least one film-forming gradient copolymer comprises at least one monomeric unit, the homopolymer of which has a Tg of less than or equal to 20° C., wherein the at least one monomeric unit is chosen from units of:
isoprene and butadiene;
methyl acrylate, ethyl acrylate, isobutyl acrylate, n-butyl acrylate, ethylhexyl acrylate, methoxyethyl acrylate, ethoxyethyl acrylate and hydroxypolyethylene glycol acrylate;
ethoxyethyl methacrylate, hexyl methacrylate, ethylhexyl methacrylate and hydroxypolyethylene glycol methacrylate;
N-(C₆₋₁₂ alkyl)(meth)acrylamides; and
vinyl esters of formula: R₃—CO—O—CH═CH₂, where R₃ is chosen from linear and branched alkyl groups comprising from 6 to 12 carbons.

67. The composition according to claim 66, wherein the N-(C₆₋₁₂ alkyl)(meth)acrylamides are N-octylacrylamides.

68. The composition according to claim 60, wherein, in formula R₃—CO—O—CH═CH₂, the alkyl groups of R₃ are chosen from vinyl neononanoate and vinyl neododecanoate.

69. The composition according to claim 1, wherein the at least one film-forming gradient copolymer comprises at least one monomeric unit, the homopolymer of which has a Tg of greater than or equal to 20° C., wherein the at least one monomeric unit is chosen from units of:
vinyl compounds of formula: CH₂═CH—R₄, wherein R₄ is chosen from a hydroxyl group; an
NH—C(O)—CH₃ group; an —OC(O)—CH₃ group; C₃ to C₈ cycloalkyl groups; C₆ to C₂₀ aryl groups; C₇ to C₃₀ aralkyl groups (C₁ to C₄ alkyl group); a 4- to 12-membered heterocyclic group comprising at least one heteroatom chosen from O, N and S; and a heterocyclylalkyl (C₁ to C₄ alkyl) group; it being possible for the cycloalkyl, aryl, aralkyl, heterocyclic or heterocyclylalkyl groups to be optionally substituted with at least one substituent chosen from hydroxyl groups, halogen atoms and linear and branched C₁ to C₄ alkyl groups wherein at least one heteroatom chosen from O, N, S and P is optionally inserted, it being possible in addition for the alkyl groups to be optionally substituted with at least one substituent chosen from hydroxyl groups, from halogen atoms chosen from Cl, Br, I, and F atoms, and from Si atoms;
acrylates of formula CH₂═CH—COOR₅, wherein R₅ is chosen from a tert-butyl group; a C₃ to C₈ cycloalkyl group; a C₆ to C₂₀ aryl group; aC₇ to C₃₀ aralkyl group (C₁ to C₄ alkyl group); a 4- to 12-membered heterocyclic group comprising at least one heteroatom chosen from O, N and S; and a heterocyclylalkyl (C₁ to C₄ alkyl) group; it being possible for the cycloalkyl, aryl, aralkyl, heterocyclic or heterocyclylalkyl groups to be optionally substituted with at least one substituent chosen from hydroxyl groups, halogen atoms and linear and branched C₁ to C₄ alkyl groups wherein at least one heteroatom is chosen from O, N, S and P is optionally inserted, it being possible in addition for the alkyl groups to be optionally substituted by at least one substituent chosen from hydroxyl groups and from halogen atoms chosen from Cl, Br, I, and F atoms, and from Si atoms;
methacrylates of formula CH₂═C(CH₃)—COOR₆, wherein R₆ is chosen from linear and branched C₁ to C₄ alkyl groups, it being possible in addition for the alkyl groups to be optionally substituted by at least one substituent chosen from hydroxyl groups, from halogen atoms chosen from Cl, Br, I, and F atoms, and from Si atoms; a C₃ to C₈ cycloalkyl group; a C₆ to C₂₀ aryl group; a C₇ to C₃₀ aralkyl group (C₁ to C₄ alkyl group); a 4- to 12-membered heterocyclic group comprising at least one heteroatom chosen from O, N and S; and a heterocyclylalkyl (C₁ to C₄ alkyl) group; it being possible for the cycloalkyl, aryl, aralkyl, heterocyclic or heterocyclylalkyl groups to be optionally substituted by at least one substituent chosen from hydroxyl groups, halogen atoms and linear and branched C₁ to C₄ alkyl groups wherein at least one heteroatom chosen from O, N, S and P is optionally inserted, it being possible in addition for the alkyl groups to be optionally substituted by at least one substituent chosen from hydroxyl groups and halogen atoms chosen from Cl, Br, I and F atoms;
(meth)acrylamides of formula: CH₂═C(R′)—CO—NR₇R₈, wherein R₇ and R₈, which may be identical or different, are each chosen from a hydrogen atom and linear and branched alkyl groups comprising from 1 to 12 carbon atoms, and R′ is chosen from a hydrogen atom and a methyl group.

70. The composition according to claim 69, wherein, in formula CH₂═CH—R₄, the heterocyclylalkyl groups of R₄ are furfuryl groups.

71. The composition according to claim 69, wherein, in formula CH₂═CH—COOR₅, the heterocyclylalkyl groups of R₅ are furfuryl groups.

72. The composition according to claim 69, wherein, in formula CH₂═C(CH₃)—COOR₆, the alkyl groups of R₆ are chosen from methyl, ethyl, propyl and isobutyl groups.

73. The composition according to claim 69, wherein, in formula CH₂═C(CH₃)—COOR₆, the heterocyclylalkyl groups of R₆ are furfuryl groups.

74. The composition according to claim 69, wherein, in formula: CH₂═C(R′)—CO—NR₇R₈, the alkyl groups of R₇ and R₈, which may be identical or different, are each chosen from n-butyl, t-butyl, isopropyl, isohexyl, isooctyl and isononyl groups.

75. The composition according to claim 1, wherein the at least one film-forming gradient copolymer comprises at least one monomeric unit, the homopolymer of which has a Tg of greater than or equal to 20° C., wherein the at least one monomeric unit is chosen from units of:
furfuryl acrylate, isobornyl acrylate, tert-butyl acrylate, tert-butylcyclohexyl acrylate and tert-butylbenzyl acrylate;
methyl methacrylate, n-butyl methacrylate, ethyl methacrylate or isobutyl methacrylate;
styrene or styrenesulphonate; and
vinyl acetate and vinylcyclohexane.

76. The composition according to claim 1, wherein the at least one film-forming gradient copolymer is present in an amount ranging from 0.1 to 60% by weight, relative to the total weight of the composition.

77. The composition according to claim 76, wherein the at least one film-forming gradient polymer is present in an amount ranging from 0.2 to 40% by weight, relative to the total weight of the composition.

78. The composition according to claim 77, wherein the at least one film-forming gradient polymer is present in an amount ranging from 1 to 35% by weight, relative to the total weight of the composition.

79. The composition according to claim 78, wherein the at least one film-forming gradient polymer is present in an amount ranging from 5 to 30% by weight, relative to the total weight of the composition.

80. The composition according to claim 1, wherein the at least one film-forming gradient copolymer is present in a form chosen from a dissolved forms and from a dispersion form chosen from aqueous and organic dispersion forms.

81. The composition according to claim 80, wherein the at least one film-forming gradient polymer is dissolved in water or an organic solvent.

82. The composition according to claim 1, further comprising at least one constituent chosen from additional film-forming polymers, additional agents which are able to form a film, water, organic solvents, thickeners, coloring materials, fillers, spreading agents, wetting agents, dispersing agents, antifoaming agents, preservatives, UV screening agents, active principles, surfactants, moisturizing agents, fragrances, stabilizing agents, antioxidants, vitamins, trace elements, basifying agents, acidifying agents, and ceramides.

83. The composition according to claim 1, wherein the composition is capable of forming a film having a damping power “tgδ” ranging from 0.5 to 1.6.

84. The composition according to claim 83, wherein the composition is capable of forming a film having a damping power “tgδ” ranging from 0.8 to 1.4.

85. The composition according to claim 1, wherein the composition is capable of forming a film having a storage modulus E′ of less than 300 MPa.

86. The composition according to claim 85, wherein the composition is capable of forming a film having a storage modulus E′ of less than 100 MPa.

87. The composition according to claim 86, wherein the composition is capable of forming a film having a storage modulus E′ less than 80 MPa.

88. The composition according to claim 1, wherein the composition is provided in a form chosen from bases for varnishes; products for making up the nails; finishing compositions and top coats to be applied to a product for making up the nails; and products for the cosmetic care of the nails.

89. A cosmetic process for making up and/or caring for nails, comprising applying to the nails a cosmetic composition, in a cosmetically acceptable medium, comprising at least one film-forming gradient copolymer comprising at least two different monomeric units and exhibiting a mass polydispersity index (PI) of less than or equal to 2.5, wherein the composition is capable of forming a film having an adhesion, measured according to Standard ASTM D 3359-7, corresponding to a detachment percentage of less than 45%.
